(19) United States
(12) Patent Application Publication
(10) Pub. No.: US 2011/0196316 A1
(43) Pub. Date: Aug. 11, 2011

(54) CATHETER

(76) Inventor: David G. Quinn, Grayslake, IL (US)

(21) Appl. No.: 13/020,086

(22) Filed: Feb. 3, 2011

Related U.S. Application Data
(60) Provisional application No. 61/302,310, filed on Feb. 8, 2010.

(51) Int. Cl. A61M 25/14

(52) U.S. Cl. 604/270

(57) ABSTRACT

A gastric/jejunal catheter comprises three major components, a triple lumen “Y” connector, a transitional “midport” bolus, and a single lumen jejunal tube with a jejunal tip. The midport bolus, in turn, comprises three lumens, a jejunal feeding lumen, a gastric relief lumen and an air lumen. The midport bolus also contains inflow and outflow ports communicating with said lumens. The bolus contains a key reinforcing arc that prevents the kinking, and resultant jejuna lumen occlusion, of the bolus.

### RELATED APPLICATIONS

This application is based on Provisional application Ser. No. 61/302,210 filed Feb. 8, 2010, and claims priority therefrom. The disclosure of this Provisional application is incorporated herein in its entirety by reference.

### FIELD OF THE INVENTION

This invention relates to catheters for use in administering fluids to body cavities, irrigating the cavities and aspirating the cavities. It relates particularly to such catheters and the distal ends thereof that contain the opening(s) for fluid egress or ingress.

### BACKGROUND OF THE INVENTION

The use of Salem sump catheters in the US for the aspiration of gastric fluid, post-surgically in gastrointestinal surgery is well documented. About 10,000 of these catheters are used annually in the USA. 50% of sump catheter tube usage is in 18Fr tubes. 25% of the usage is in 16Fr tubes. The remaining usage is spread in decreasing amounts over 14fr, 12Fr, 10Fr and 9Fr tubes. The vast majority of these tubes utilize polyvinyl chloride as the tube material, commonly referred to as PVC. The use of PVC requires large wall thicknesses and therefore correspondingly large French (Fr) sizes to support internal lumens. The existing sump tubes also incorporate conventional, easily clogged, inflow ports that have not been changed since the early 1970's. All of the existing postsurgical sump tubes are contraindicated for enteral feeding because of flow port clogging.

Recent clinical studies have shown that patients in the immediate post surgical intensive care units should be fed immediately after surgery. It is not possible to immediately enterally feed any of the patients now being given Salem sumps because none of these patients have peristalsis and cannot, therefore, empty their stomachs. Stomach contents must be continually be aspirated. These clinical studies show that if patients are fed immediately fed after surgery they are released 2.2 days earlier than patients not being fed. Immediate feeding also results in a 55% reduction rate in post-surgical infections.

Many postsurgical patients, including all of those with impaired peristalsis, must be fed deep in the jejunum, not in the stomach. Clinical studies have also shown that feeding deep in the jejunum does not stimulate the secretion of enzymes in the duodenum. However, deep jejunal feeding does stimulate the secretion of gastric juices, hence the critical need to aspirate the stomach simultaneously while feeding into the jejunum. Deep jejunal feeding does not stimulate enzyme secretion in the duodenum and therefore deep jejunal feeding must be accomplished with predigested elemental diets, not the normal undigested polymeric diets.

### SUMMARY OF THE INVENTION

This invention is embodied in a new midport catheter that incorporates improved inflow and outflow ports in a midport bolus in the stomach. The catheter provides for much larger, effectively protected ports to prevent clogging and to maintain aspiration flow. The catheter also provides for the feeding of feeding formulas deep into the jejunum through a jejunal catheter lumen with an improved outflow port.

The catheter invention covers all the size requirements of existing gastric sump devices as now represented by the size availability of Salem sumps. The size disclosed in this patent application is 18Fr. The invention will also cover 16Fr, 14Fr, 12Fr and 10Fr sizes, all of which have the same design configurations as the 18Fr version. Because of the catheter tube lumen designs and because of the utilization of stronger polyurethane, a midport catheter incorporating a third lumen for feeding in the jejunum can be constructed that provides the third lumen for feeding while at the same time providing larger air vent lumens and gastric aspiration lumens per French size than existing Salem sumps. The 18Fr tube has an outside diameter (OD) of 0.242″. The actual midport bolus has an OD of 0.278″ or 20Fr. This size differential between the tube and midport bolus is not important because the tube is initially inserted through the nose via a 12Fr jejunal tube. The most important portion of the tube is the portion of the 18Fr length that exits the patient's nose. The length of the midport bolus is 1.08 inches. Normal enteral feeding tubes for adult and juvenile use are 12Fr, 10Fr, 8Fr, and 6Fr. The jejunal tube leading from the midport bolus covers these sizes and are as follows: 18Fr midport bolus/12Fr jejunal line; 16Fr midport bolus/10Fr jejunal line; 14Fr midport bolus/9Fr jejunal line; 12Fr midport bolus/8Fr jejunal line; and 10Fr midport bolus 6Fr jejunal line.

To both reduce costs and to allow the midport bolus to be better secured to the thin walls of triple lumen tube, the midport bolus is formed by being overmolded directly to the tube that is cut at a 45 degree angle. The tip bolus of the jejunal tube is overmolded over a tube that is skived at a level slightly below the internal radius of the tube. The midport bolus incorporates a reinforcing arc which extends from the point on the bolus where the 45° skived gastric port begins and terminates at the point that the flow port recess in the bolus transitions up to the outside diameter of the bolus.

An object of the invention is to provide a new and improved midport catheter bolus construction, a construction which allows for the elimination of bolus port side walls, where the gastric/air vent port is recessed with a port that has an effective recessed level of the full OD of the bolus.

Another object of the invention is to provide a gastric/air vent port whereby both the gastric lumen and the air vent lumen of the triple lumen tube are terminated at the same point at the distal end of the tube so that increases in suction pressure are instantly relieved because of the proximity of aspiration lumen port and the vent line port.

Another object is to maintain direct access between the terminus of the gastric lumen and the adjacent terminus of the air vent lumen.

Yet another object of the invention is to provide a recessed portion for protection of outflow and inflow that extends around 270° of the circumference of the first bolus.

Another object of the invention is to provide smooth recessed grooves in the distal bullet tip of the first bolus that create for flow channels in the tip which allow flow communication from the distal end of the bolus to the 270° recessed area so as to communicate with the gastric aspiration lumen and the air vent lumen.

Another object of the invention is to provide a distal end 45° skiving of the three lumen tube to provide methodology for the overmolding of the main midport bolus to the thin walled tube which prevents leaking between lumens.

Still another object of the invention is to provide a NGJ catheter that is the smallest size possible while at the same time providing adequate ingress and egress of fluid and air from both the stomach and the jejunum.

### BRIEF DESCRIPTION OF THE DRAWINGS

The invention, including its construction and method of operation, is illustrated more or less diagrammatically in the drawings, in which:

FIG. 1 is a side view of the entire gastro/jejunal catheter, including a three port “Y” connector, the transitional gastric bolus and the jejunal bolus;

FIG. 2 is a side view of a gastric/jejunal catheter including a gastric transitional bolus, showing the bolus connected to both the gastric and the jejunal tube;

FIG. 3 is an opposite side view of a gastric/jejunal catheter including a gastric transitional bolus, showing the bolus connected to both the gastric and the jejunal tube;

FIG. 4 Is a top plan view of the catheter of FIG. 2;

FIG. 5 is a bottom plan view of the catheter of FIG. 2;

FIG. 6 is a longitudinal view of the catheter taken along lines 6-6 of FIG. 3;

FIG. 7 is an enlarged view of a partial portion of FIG. 6.

FIG. 8 is an enlarged end perspective view of FIG. 2 taken along lines 8-8;

FIGS. 9, 10, 11, 12, 13 14 and 15 sectional views taken along lines 9-9, 10-10, 11-11, 12-12, 13-13, 14-14, and 15-15 of FIG. 3;

FIG. 16 is the side view of FIG. 2 and the sectional view of FIG. 9 showing the “phantom” flow path of the jejunal lumen through the catheter;

FIG. 17 is the top plan view of FIG. 4 and the sectional view of FIG. 9 showing the “phantom” flow path of the jejunal lumen through the catheter;

FIG. 18 is the side view of FIG. 2 and the sectional view of FIG. 9 showing the “phantom” flow path of the gastric aspiration lumen through the catheter;

FIG. 19 is the top plan view of FIG. 4 and the sectional view of FIG. 9 showing the “phantom” flow path of the gastric aspiration lumen through the catheter;

FIG. 20 is the side view of FIG. 2 and the sectional view of FIG. 9 showing the “phantom” flow path of the gastric vent lumen through the catheter;

FIG. 21 is the top plan view of FIG. 4 and the sectional view of FIG. 9 showing the “phantom” flow path of the gastric vent lumen through the catheter;

FIGS. 22, 23, 24, 25 and 26 are sectional views of FIG. 9 showing the 18fr, 16fr, 14FR 12FR and 10fr showing relative dimensions.

### DESCRIPTION OF THE PREFERRED EMBODIMENTS

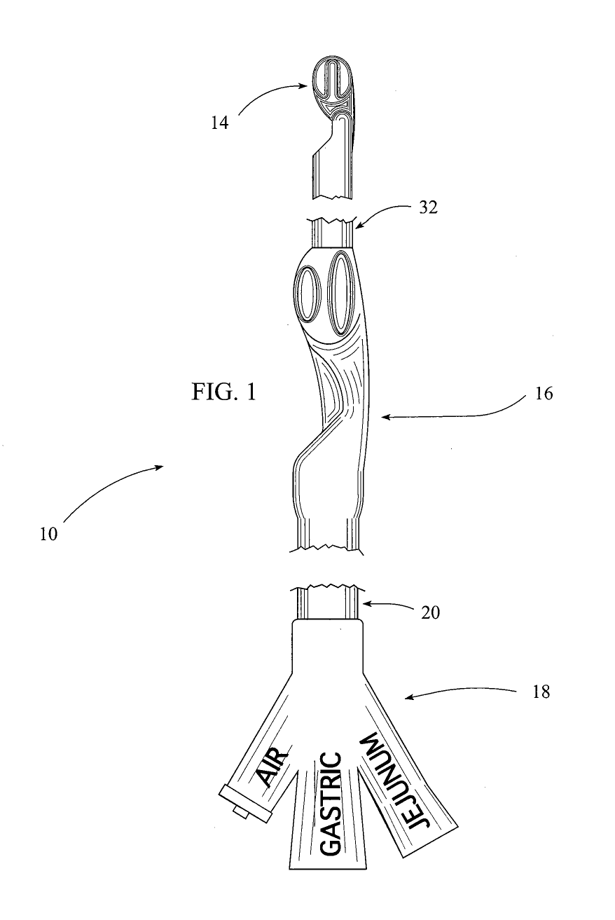

Referring now to the drawings (especially FIGS. 1, 2, 3, 4 and 5), in FIG. 1 the three major components of a gastric/jejunal catheter 10 are shown. The catheter 10 comprises a triple lumen “Y” connector 18, a multiple lumen tube 20, a transitional (midport) bolus 16, a single lumen jejunal tube 32 and a jejunal tip 14.

In FIG. 2 an 18Fr triple lumen tube is shown at 20. This segment of tube for adult or juvenile uses can be either 36″ or 42″ long, depending on the size of the patient. The goal is to place the bolus in close proximity to the pylorus so that the stomach can be aspirated at its emptying point.

The tube 20 size disclosed is an 18Fr, triple lumen, tube with an outside diameter of 0.242.″ The midport bolus 16 is 1.08″ in length. The jejunal tube 32 exiting the distal end of bolus 16 is a 12Fr, single lumen tube, with an outside diameter (OD) of 0.164″. Its length is 42′. Therefore, the full length is either 76″ or 80.″ Other sizes are 16/10Fr, 14/9Fr, 12Fr/8Fr and 10/6Fr.

The outside diameter of the bolus 16 is 0.278″, reflecting over-molding walls of 0.018″ shown at over molding area 23 in FIG. 2. The lowest portion of ramp 21, as calculated from the outside diameter of bolus 16, is recessed 0.124″ or slightly more than the radius 0.121″ of multi lumen tube 30. The side ramp 26 is recessed 0.080″ from the 0.278″ outside diameter of bolus 16. The cross-sectional view of the approximate lowest recessed level of ramp 21 is shown in FIG. 12. The 0.080″ deepest recess level of side ramp 26 is shown in FIG. 13.

In FIG. 2 the socket area 23 encloses the triple lumen tube 20 where it is overmolded on the tube. The top of the ramp 21 leading from first gastric aspiration lumen 38 leads to distal end bolus tip 34. The bottom, recessed groove level, of air vent lumen 48 is shown at 22. The continuation of side recess area is shown at 26.

As shown in FIGS. 2, 3, 4 and 5, this recess 26 continues the full side of the bolus 16. The grooved side flow channels in distal tip 34 are shown at 28 and 30. The leading top edge of the gastric lumen 38 is seen at 25.

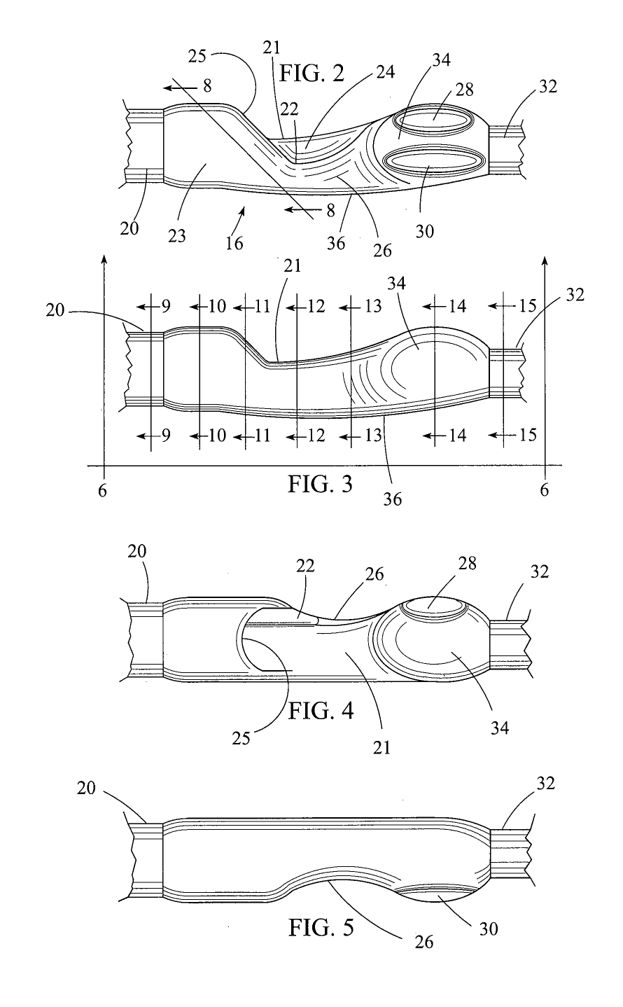

FIG. 3 shows a side view of the bolus 16 opposite to that shown in FIG. 2. FIG. 4 shows a tip view of the bolus 16. The lower edge of the vent port is shown at 22. The recessed area of the bolus blends from 22 and is shown at 26. FIG. 5 shows the bottom of the bolus 16. The recess on the side of the bolus is shown at 26.

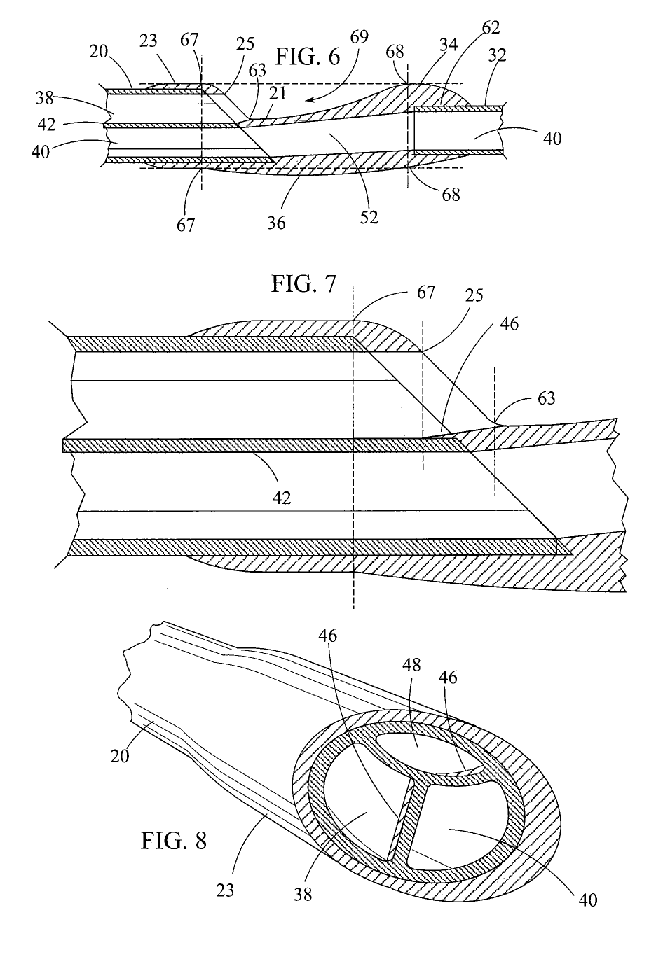

FIG. 6 shows a cross-section of the bolus 16 at section 6. 42 is the cross section of the central septum separating gastric lumen 38 and jejunal lumen 40. The 45° skived leading edge of multi-lumen tube 20 is shown at 25. Shown at 52 is the jejunal lumen in the bolus 16. Shown at 40 is the round jejunal lumen in the 12Fr single lumen jejunal line 32.

FIG. 7 is an expanded view of the area 23 of FIG. 6. A slight overmolding 46 assists in the adhesion of the bolus 16 to the septum 42. FIG. 8 shows the slight overmolding 46 in both the gastric lumen 38 and the air lumen 40.

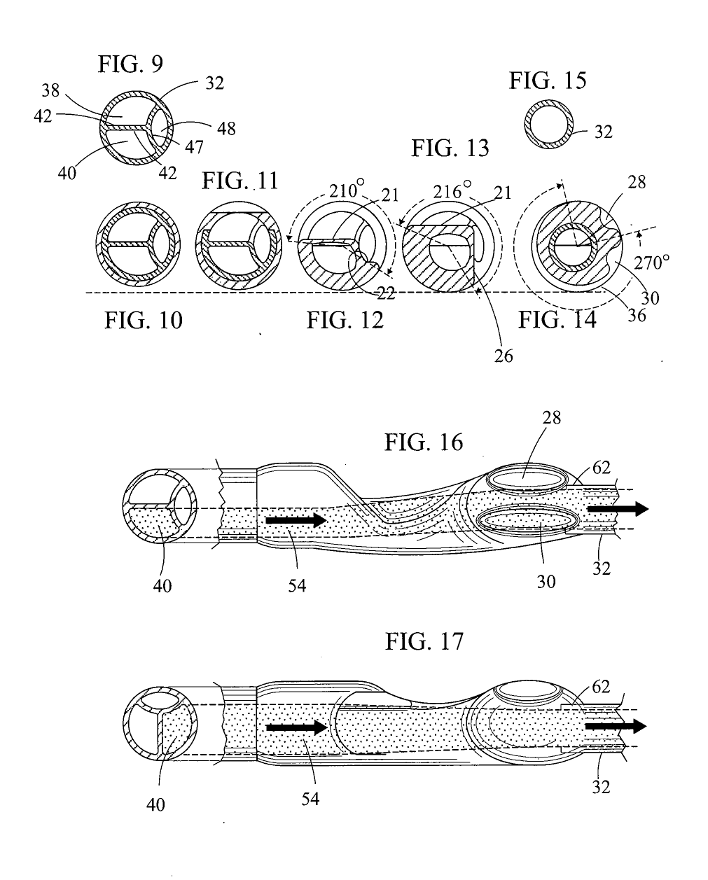

FIGS. 9-15 show cross-sections of the catheter 10 seen in FIG. 3. FIG. 13 also shows the air lumen bottom portion 22 as it transitions distally. FIG. 13 shows the transition of the recess 26 where it forms a full 180° recess 26 on the side of the bolus. Note the full transition of side recess 26 to tip recess/ramp 21 forming a full 270° recess.

The FIGS. 6 and 7 illustrate positioning the reinforcing arc 36. This element is critical because it prevents the entire overmolded bolus 16 from kinking and occluding the jejuna lumen 40. It is also important that this bolus does not add effective outside diameter to the bolus. Why is that true? Because in FIGS. 6 and 7 the proximal end of the bolus originates at points 67 where the recess 69 begins. As the bolus is inserted through the mucosa of the nose, nasopharynx and eosophagus, the tissue is not stretched beyond the overall outside diameter of the largest OD of the bolus. At its distal end the reinforcing arc ends at the point 68 where it meets the portion of the OD of the leading portion of the bolus 16. It is because the part would become weaker if the reinforcing begins or terminates before the OD of the bolus. The length of the reinforcing arc 36 extends from the point on the first bolus where the recessed ramp begins at the top of the 45 degree skived gastric port and extends to the point where the ramp meets the leading portion of the bolus at the outside diameter of the bolus as defined by the portion of the bolus that is over molded over the three lumens of the three lumen tube at its distal end; (FIGS. 6, 67 to 68). Without the reinforced arc the tube will kink at the point 63. Because of the 45 degree skive the arc can begin before point 63 without actually making the effective OD of the bolus larger. The arc 36 is protecting from bending the single lumen portion of the bolus.

Another feature is the fact that we describe that the deepest part of the recess is slightly larger than the radius of the actual three lumen tube. The configuration of the triple lumen extrusion and the reinforcing arc allows this depth.

FIGS. 12, 13, and 14 are also instructive. The reinforced arc 36 is enlarged beyond its shape defined by the ramp 21 because the portion of the bolus that contains the air vent lumen 48 is utilized as an additional side portion of the overall reinforced arc, basically expanding the arc beyond the approximate 180 degrees. FIG. 12 shows the recessed space at 210 degrees. The recess tapers to 216 degrees in FIG. 13. In FIG. 14 the bottom taper of the leading portion 34 of the bolus 16 tapers away from the reinforcing arc 36, essentially creating a 270-degree recession.

The grooved side flow channels 28 and 30 provide for flow access from the front, distal end of the bolus 16 to the recessed space that communicates with the gastric lumen 38 and the air vent lumen 48. Flow to the recessed space is from the top, sides and front of the bolus. The recessed area transitions from 210 degrees around bolus 16 at the point where the gastric and air vent lumens access the recessed area in cross section 12-12 to 270 degrees at the point where the distal end of the bolus meets the two flow channels 28 and 30 that provide flow access from the leading distal portion of the bolus 16.

Now referring to FIGS. 16, 17, 18, 19 20 and 21. All of these FIGs. utilize the tube cross section of multi-lumen tube 20 that is shown in FIG. 9. This tube cross section is matched with the side and top plan views of FIGS. 2 and 3 to show “phantom” flow through the bolus, and in the case of the jejunal line, through the bolus 16 and the jejunal single lumen tube 32.

FIGS. 16 and 17 show the flow and direction of the jejunal flow. In FIG. 16 the semi-D lumen 40 tapers to become larger in this view as it tapers to a circular shape in tube 32. In the top view of FIG. 17, the flow taper becomes more restricted as it tapers to a full circular shape. This flow channel is formed by a molding pin that enters the distal end of bolus 16 and extends the full length of the bolus and into the jejunal lumen 40. At this point the molding forms a socket 62 for the jejunal single lumen tube 32. In normal molding this molding pin (not shown) would present an “undercut” and the pin could not be removed from the part. However in this case the pin can easily be removed from the part because the taper pulls easily through and from the flexible part without damaging it.

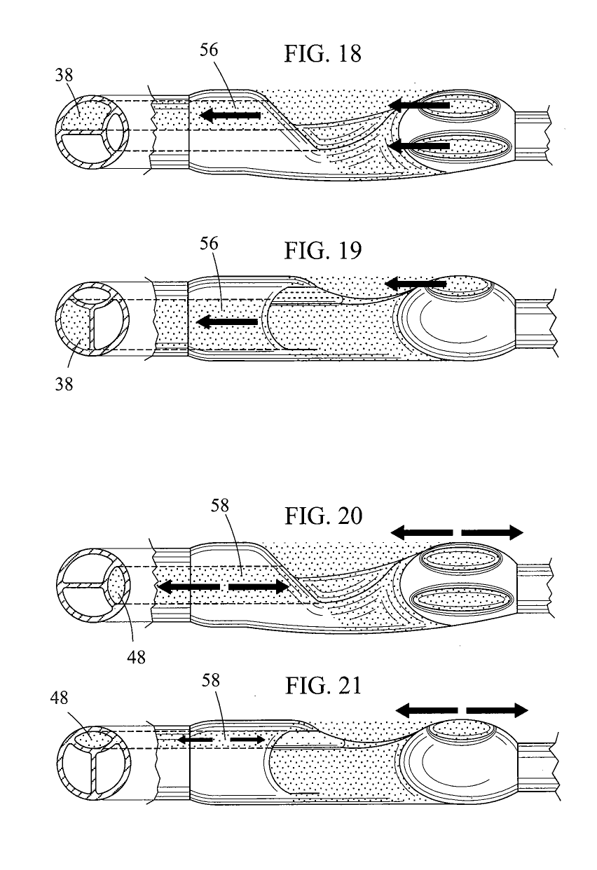

FIGS. 18 and 19 illustrate the internal and exterior flow in phantom of the gastric line. The flow through the flow grooves 28 and 30 communicate with the flow from gastric port 38. FIGS. 20 and 21 show the phantom flow through air lumen 40.

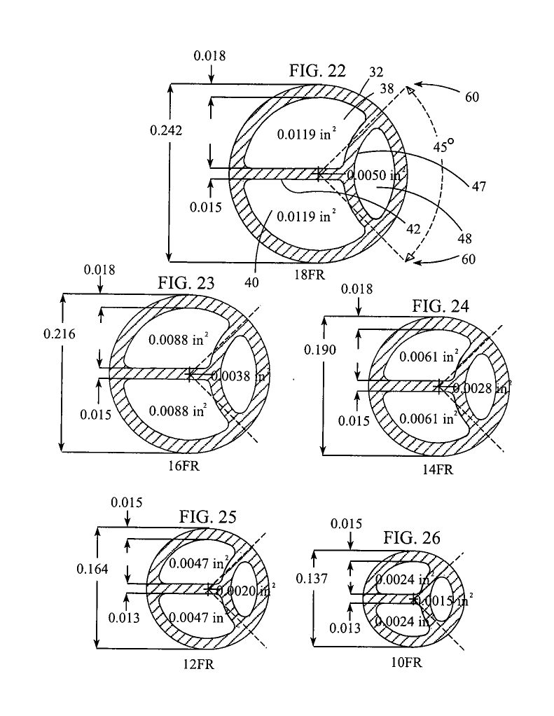

Now referring to FIGS. 22, 23, 24, 25 and 26. These FIGs. show the dimensions of the five multiple lumen tube sizes for the midport bolus 16. FIG. 22 shows the 18fr tube described in this application. The configurations of the other sizes are smaller versions of the 18Fr tube 20. The “line” 60 is a tangent extending from the center of tube at the middle of internal septum 42. Therefore these lines are true tangents. These lines form tangents that are at 45° from the center of the tube. All of the arcs forming the air vent lumen 40 and separating it from lumens 38 and 40 fall inside the width of the septums 42 where they meet and attach to the outside wall of the tube 32. This combination of the septum arc forming the air vent lumen 48 form a strengthening “member” inside the main tube that assists in preventing the tube from collapsing or kinking. In addition, the fact that all three of the internal septum points of joining the outside wall of tube are at 45° angles minimize the amount of normal “filling” that occurs at the junctures of internal septums and the outer wall of a tube. This filling reduces the size of the internal lumens. It is virtually impossible to extrude polyurethane to form a true unfillited juncture of an internal septum and the outer wall.

To review the functional aspects of the present inventions, they embody an enteral catheter that provides access to both the stomach and the deep jejunum for feeding, aspiration and decompression. The catheter includes a triple lumen tube that joins to a triple lumen “Y” connector at the proximal end of the tube. The connector serves the three lumens as a source for venting air, for fluid aspiration and for fluid infusion. The gastric aspiration lumen, the jejunal feeding lumen and the air vent lumen all connect to a transitional midport connector bolus in the stomach at the distal end of the three lumen tube.

The gastric lumen and the air vent lumen both open into the stomach through a common gastric port. The jejunal lumen communicates with jejunal lumen in the midport bolus. Midport jejunal lumen transitions from a modified “D” shape to a full circle shape. The latter provides for the attachment of a smaller, round single lumen tube that extends to the jejunum. The gastric lumen and the air vent line terminate at the same point, side-by-side, into the common gastric port. The gastric port is recessed to the level of its full internal lumen, thereby providing a recess for maximum protection against occlusion and maximum area for outflow. This recessed level is at the top of the mid-tube septum that separates the gastric tube lumen and the jejunal lumen and is therefore recessed to almost the internal radius of the triple lumen tube. This septum transitions to become the ramp of the recessed port that serves both the gastric port and the air vent port. The air vent lumen continues distally in its same shape until it transitions into another recess on the side of the midport bolus that also provides protection against occlusion and is source of flow. This side recess extends around the full 180 degree side of the midport bolus. The gastric lumen, the vent lumen, the top recess and the side recess all communicate with each other, thus providing 270 degree access to the gastric and air vent lumen for flow in or out of the main three lumen tube and also flow in or out from the loading distal portion of the bolus 10 through flow channels 28 and 30.

It is intended that the foregoing detailed description be regarded as illustrative, rather than limiting. It is to be understood that it is the following claims, including all equivalents, that are intended to define the spirit and scope of this invention.

What is claimed is:

1. A catheter for delivering fluid into, or aspirating fluid out of, a body cavity or cavities, comprising:
a) a multiple lumen tube containing a first lumen, a second lumen and a third lumen and having a proximal end and a distal end, said tube containing a septum separating said first lumen and second lumens and a septum separating first and second lumens from said third lumen, said first and third lumens being formed so that first lumen and third lumen are the same length and open at the same distal point, said second lumen being formed so that said lumen is longer than said first and second lumen and both said septums terminate at the distal ends of first and third lumens, and said second lumen terminates at a predetermined distance from where said first and third lumens open at said distal end of multiple lumen tube;
b) a first bolus having a nose end and a connector end and an axial passage therethrough;
c) said first bolus being formed independently of said multiple lumen tube and said distal end of multiple lumen tube being connected to said multiple lumen tube over axial passages of said first, second and third lumens;
d) a single lumen catheter tube separate from said multiple lumen tube and seated in said axial passage of said first bolus, said single lumen tube having a port in its distal end; and
e) a second bolus on the distal end of said single lumen catheter tube;
f) said port in said distal end of said single lumen tube being formed in the side of said second bolus.

2. The catheter of claim 1, further characterized in that:
a) said first bolus contains a reinforcing arc formed on one side thereof and extending axially of said first bolus;
b) the arc extends from the beginning of the recessed flow area over the ramps; and
c) the reinforcing arc terminates at the point where the recessed blow area transitions up to meet the outside diameter of the bolus.

3. A catheter, comprising:
a) a multiple lumen tube;
b) a first bolus formed independently of said multiple lumen tube, said bolus being connected to said distal end of said tube, said bolus forming at least a portion of a each of a first lumen port extending radially of said catheter over said substantially recessed outer wall, said first port and communicating with first said lumen, and a second port from lumen three communicating with said first port,
b) said first lumen extending to an opening at a predetermined distance from said distance from said distal end of multiple tube; and
c) said bolus including an attachment section fastened to said septum where if comprises and outer wall and has a rear face defining a ramp including a surface inclined at an angle to said septum.

4. The catheter of claim 3, further characterized in that:
said ramp extends rearwardly to an intersection with first lumen opening.

5. The catheter of claim 3, further characterized in that:
said catheter tube contains a third lumen;

6. The catheter of claim 3, further characterized in that:
a) the third lumen forms a recessed ramp on the side of the first bolus; and
b) the said ramp transitions distally to the end of the first bolus.

7. The catheter of claim 3, further characterized in that:
a) the recessed top ramp of the first lumen and the side recessed ramp of the third lumen communicate with each other;
b) the said communicated recessed ramp form a recessed ramp for flow and aspiration around 270° of the circumference of the first bolus.

8. The catheter of claim 3, further characterized in that:
a) the first and third lumens of the multiple lumen catheter tube terminate at the same distal point;
b) both said first and third lumens are adjacent to each other;
c) both said first and third lumens communicate with the 270° top and side recessed ramp.

9. The catheter of claim 3 further characterized in that:
a) the 270° degree recessed ramp in the first bolus surrounds the second lumen that extends to the distal end of the single lumen tube;
b) the portion of the second lumen contained in the first bolus transitions from semi “D” shape to a circular shape at the distal end of the said bolus.

10. The catheter in claim 3 further characterized:
a) the first midport bolus ramp serving the gastric aspiration lumen and secondarily the vent lumen is recessed at its most recessed point relative the outside diameter of the bolus to a depth that is slightly larger than the radius of the multi-lumen tube.

11. The catheter in claim 3 further characterized:
a) in that the side ramp serving primarily the vent lumen and secondarily the gastric lumen of the bolus is recessed to a depth that is one third the radius of the multi lumen tube.

12. The catheter of claim 3 further characterized in that:
the multi-lumen is skived at its distal end at a 45° angle to the mid line middle septum that separates the first and second lumens.

13. The catheter of claim 3 further characterized in that:
all of the attachment intersections of the four points whereby the internal lumen septum intersect with the outer main tube wall are at approximately 45 degree tangents to the outer wall.
